(19) United States
(12) Patent Application Publication
(10) Pub. No.: US 2024/0366682 A1
(43) Pub. Date: Nov. 7, 2024

(54) PROCESS FOR THE PREPARATION OF A PULMONARY SURFACTANT

(71) Applicant: CHIESI FARMACEUTICI S.P.A., Parma (IT)

(72) Inventors: Tomaso GUIDI, Parma (IT); Luca RIZZO, Parma (IT); Monica BOCCHI, Parma (IT)

(73) Assignee: CHIESI FARMACEUTICI S.P.A., Parma (IT)

(21) Appl. No.: 18/576,170

(22) PCT Filed: Jul. 4, 2022
(86) PCT No.: PCT/EP2022/068366

§ 371 Date: Jan. 3, 2024

(30) Foreign Application Priority Data
Jul. 5, 2021 (EP) 21183790.1

(51) Int. Cl. A61K 35/42; A61K 9/00; A61K 47/10

(52) U.S. Cl. CPC A61K 35/42; A61K 9/0082; A61K 47/10

(57) ABSTRACT

This invention is directed to a process for the preparation of an exogenous pulmonary surfactant, in particular a modified natural surfactant or a reconstituted surfactant, wherein the final filtration step is performed without the use of chlorinated solvents. The invention is also directed to the product as obtained by said process, and to the corresponding pharmaceutical compositions.

### FIELD OF THE INVENTION

This invention is directed to a process for the preparation of a modified natural surfactant. The invention is also directed to the product as obtained by said process, to the corresponding pharmaceutical compositions and uses thereof.

### BACKGROUND OF THE INVENTION

Pulmonary surfactant is a lipid-protein mixture that coats the inside of the alveoli. The presence of the lipids as a monolayer at the air-liquid interface in the alveoli reduces the surface tension. Thereby it diminishes the tendency of alveoli to collapse during expiration and perhaps also reduces the transudation of fluid into the air spaces.

Endogenous pulmonary surfactant contains about 80% weight phospholipids, 10% weight proteins, and 10% weight neutral lipids such as triglycerides and further minor components.

Among the phospholipids, a role of paramount importance plays the disaturated form dipalmitoyl phosphatidylcholine (DPPC) as it forms a monomolecular film at the air-liquid interface during the inspiration phase, thereby stabilising the alveolar system.

At least four proteins are typically present in natural pulmonary surfactants, SP-A, SP-B, SP-C and SP-D. Of these four, SP-B and SP-C are distinct, low molecular weight, hydrophobic proteins that have been shown to enhance the surface-active properties of surfactant phospholipid mixtures, presumably by facilitating transfer of lipids from the bulk phase lamellar organization to the air-water interface and also by stabilizing the lipid monolayer during expiration (see Hawgood S et al Biochim Biophys Acta. 1998 Nov. 19; 1408 (2-3) pp 150-160; Johansson J Biochim Biophys Acta. 1998 Nov. 19; 1408 (2-3) pp 161-72).

Pulmonary surfactant deficiency or dysfunction causes a severe respiratory disease named respiratory distress, which is the cause of high morbidity and mortality, particularly in pre-term infants and in adults affected by various pathologies involving a severe pulmonary insufficiency.

Replacement therapy with a variety of exogenous surfactants has proved beneficial in both experimental and clinical studies.

In particular, modified natural surfactants extracted from mammalian lungs are widely used as substitutive therapy.

Widely utilized modified natural surfactants are poractant alfa derived from porcine lung, and sold under the trademark of Curosurf® (Chiesi Farmaceutici SpA, Italy, beractant (Survanta®, AbbVie Inc, USA) and bovactant (Alveofact®, Lyomark Pharma GmbH, Germany), both derived from bovine lung, and calfactant (Infasurf®, Ony Biotech, USA) derived from calf lung.

In general, surface-active material is removed from the airways of mammalian lungs by different approaches, then followed by extraction with organic solvents, and drying.

The raw material obtained upon drying is defined hereinafter as the paste.

For example, the process for obtaining calfactant was disclosed in U.S. Pat. No. 6,129,934, where the surface-active material is removed from the airways of calf lungs by the process of bronchoalveolar lavage. The fluid collected by bronchoalveolar lavage is centrifuged to collect a concentrated sediment of surface-active material. The sediment of surface-active material is then brought to desired volume by addition of an aqueous solution, such as physiological saline.

The extraction of surface-active material from the aqueous suspension may be carried out using various organic solvents, including, but not limited to, chloroform, benzene, and mixtures of chloroform and methanol, ether and ethanol, and hexane and ethanol. In a preferred embodiment, a mixture of chloroform and methanol is used.

The process for obtaining beractant instead is disclosed in U.S. Pat. No. 4,397,839 and comprises the steps of (a) bringing the minced lung tissue of a mammal into contact with an electrolyte solution to obtain an extract; (b) centrifuging the extract to collect a crude sediment; (c) adjusting the specific gravity of an aqueous suspension of the crude sediment by the addition of sodium chloride and centrifuging the adjusted suspension to isolate a top layer comprising an emulsified scum layer; (d) dialyzing an aqueous suspension of the top layer and lyophilizing the dialyzed suspension to obtain a crude dry product; (e) bringing the crude dry product into contact with an acetic acid ester to collect a material insoluble in the acetic acid ester and then bringing the insoluble material into contact with an organic solvent mixture to obtain a purified filtrate; and (f) concentrating the purified filtrate to obtain a solid residue

The process of preparation of poractant alfa is disclosed in EP 286,011, and comprises the following steps: i) mincing the animal lungs and washing them in a saline solution; ii) separating the surfactant by means of subsequent filtration, centrifugation and extraction with a mixture of chloroform:methanol 2:1 (v/v); iii) recovering the raw lipid fraction through evaporation of the solvent to dryness; iv) purifying the latter one by reversed phase chromatography using the resin LipidexR-5000, with 1,2-dichloroethane:methanol 1:4 (v/v) as eluant.

The purification step by chromatography column may be critical for ensuring a modified natural surfactant having a well-defined, reproducible composition and it has been optimized in order to obtain a surface active material enriched in the constituents considered responsible of the biological activity, i.e. the polar lipids, mainly phospholipids and the hydrophobic proteins SP-C and SP-B, and substantially rid of the unessential components i.e. carbohydrates, neutral lipids such as triglycerides, cholesterol and free fatty acids.

Due to the needs in improving the surfactant preparations, synthetic surfactants mimicking the composition of the modified natural surfactants have been developed. Said synthetic surfactants are known as reconstituted surfactants.

Examples of reconstituted surfactants include, but are not limited to, lucinactant (Surfaxin™, Windtree Therapeutics, Inc., Warrington, Pa.) and elifactant, the product having the composition disclosed in the paragraph abridging page 9 and page 10 of WO 2010/139442 and known in the art with the experimental code CHF 5633.

Independently on the type of surfactant, the paste of modified natural surfactants or the components of the reconstituted surfactants are typically dissolved and subjected to a further step of purification by sterilizing filtration.

For example, according to EP 286,011, the paste before filtration could be dissolved in 98:2 chloroform/methyl alcohol (V/V).

However, the use of particular solvents such as chloroform may have some impacts on both the remaining steps of the manufacturing process and potentially the quality of the product. For instance:
i) both piping and filters are usually exposed to a chemical stress with a possible reduction of the range of material which can be used;
ii) by consequence, the level of extractable components into the product may need an additional treatment;
iii) the process of the solvent evaporation step may take some time or multiple repetitions in order to strip out the residual presence of these chemical components.

Therefore, an alternative step involving a different solvent would be desirable to reduce or mitigate the above risks and by assuring, at the same time, all the quality attributes of the product.

On the other hand, due to the hydrophobic structure of the phospholipids, it is quite challenging performing a dissolution phase in an aqueous media. Thus, new candidate should still be an organic solvent meeting some defined criteria as:
i) be replaceable to chloroform;
ii) due to the presence of temperature-sensitive components such as proteins, capable of dissolving the surface-active material around the body temperature.
iii) allowing a similar proportion of the volumes at stake for the dissolution phase;
iv) being suitable for the evaporation phase, namely having the proper physical properties (boiling point, vapor pressure, etc.);
v) being readily available with reduced or no risk of phase out on the market.

The problem is solved by the present invention.

### SUMMARY OF THE INVENTION

In a first embodiment, the invention provides a process for the preparation of an exogenous pulmonary surfactant selected from modified natural surfactants or reconstituted synthetic surfactants, comprising the steps of:
i) extracting said modified natural surfactant from mammalian lung in form of dry paste or mixing the dry components of said reconstituted synthetic surfactant;
ii) purifying the obtained paste or mixture;
wherein the step ii) of purification is performed by:
iii) dissolving the paste of the modified natural surfactant or the components of the reconstituted surfactant in an organic solvent comprising 2-methyl-2-propanol,
iv) subjecting the obtained solution to sterilization by filtration; and
v) drying;

Advantageously, the dry paste of the modified natural surfactant may be obtained from mammalian lung by lavage or column elution.

Preferably the modified natural surfactant is poractant alfa.

Therefore, in a preferred embodiment, the invention provides a process for preparing poractant alfa, said process comprising the steps of:
i) mincing the pig lungs in a blender;
ii) extracting the minced lungs with a physiological solution, then filtering and subjecting the mixture to centrifugation;
iii) extracting the supernatant with an organic solvent or mixtures thereof then evaporating the organic phase to dryness;
iv) separating the fractions containing the polar lipids and the hydrophobic proteins SP-C and SP-B;
v) collecting and pooling said fractions then evaporating the organic solution to dryness to yield the surfactant in the form of dry paste;
vi) dissolving the obtained paste in an organic solvent comprising 2-methyl-2-propanol;
vii) subjecting the obtained solution to sterilization; and
viii) drying;
The present invention is also directed to an exogenous pulmonary surfactant as obtained by the process of the invention.

In another aspect, the invention is directed to pharmaceutical formulations comprising, as active ingredient, the exogenous pulmonary surfactant obtained by the process of the invention.

The invention is also directed to the exogenous pulmonary surfactant as obtained by the process of the invention, for use for the prevention or treatment of a variety of pulmonary disorders, abnormal conditions and diseases caused or related to a deficiency or dysfunction of the pulmonary surfactant.

In a further aspect, the invention is directed to the use of the exogenous pulmonary surfactant as obtained by the process of the invention in the manufacture of a medicament for the prevention or treatment of a variety of pulmonary disorders, abnormal conditions and diseases caused or related to a deficiency or dysfunction of the pulmonary surfactant.

Yet, the invention provides a method for the prevention or treatment of a variety of pulmonary disorders, abnormal conditions and diseases caused or related to a deficiency or dysfunction of the pulmonary surfactant, said method comprises administering to a patient in need of such treatment a therapeutically effective amount of the of the exogenous pulmonary surfactant as obtained by the process of the invention.

### FIGURE

FIG. 1 shows the results in terms of tidal volumes (ml/kg) as a function of time/pressure of Curosurf® as obtained by the process of the invention versus Curosurf® control and untreated animals.

### DEFINITIONS

The terms “surfactant” and “surface active material” are used as synonymous.

For an exhaustive definition of the different types of exogenous pulmonary surfactants-see Wilson D. Expert Opin Pharmacother 2001, 2, 1479-1493.

The term “modified natural surfactant” means a lipid extract of minced mammalian lung which, due to the lipid extraction step used in the manufacture process, is deprived of the hydrophilic proteins SP-A and SP-D and contains variable amounts of the hydrophobic proteins SP-B and SP-C. Depending on the method of extraction, may contain different amounts of phospholipids, non-surfactant lipids and other minor components.

The term “artificial” surfactant” means a simple mixture of synthetic compounds, primarily phospholipids and other lipids that are formulated to mimic the lipid composition and behaviour of natural surfactant, but devoid of surfactant proteins,

The term “reconstituted” pulmonary surfactant means an artificial pulmonary surfactant to which have been added pulmonary surfactant proteins/peptides isolated from animals or proteins/peptides manufactured through recombinant technology.

The term “dry” refers to the surfactant material as obtained after evaporation of the organic solvents. It contains limited amounts of residual solvents including water.

The alcohol 2-methyl-2-propanol is also known as tert-butanol.

The term “polar lipids” include mainly phospholipids and some other minor components such as plasmalogens, glycolipids, cardiolipins, and lysophospholipids.

The term “phospholipids” refers to lipids that are composed of a nonpolar hydrophobic tail, a glycerol or sphingosine moiety, and a polar head. The nonpolar hydrophobic tail is usually a long chain fatty acid which in turn can be saturated (e.g. myristic, palmitic and stearic acid), monounsaturated (e.g. oleic acid) or polyunsaturated (e.g. linoleic and arachidonic acid).

The polar head has a phosphate group that is attached to a nitrogen-containing base.

The phospholipid fraction in the surfactant is mainly composed of phosphatidylcholine (PC), phosphatidylinositol (PI), phosphatidylethanolamine (PE), phosphatidylserine (PS), phosphatidylglycerol (PG), and sphingomyelin (SM).

The term “neutral lipids” include triglycerides, diglycerides and monoglycerides. The term “size exclusion liquid-gel chromatography” refers to a chromatographic system where the stationary phase is a gel which retains the substances to be separated according to their molecular size.

The term “biosimilar of poractant alfa,” refers to a modified natural pulmonary surfactant which has the same safety profile, it is therapeutically equivalent, it has a similarity in the quali-quantitative composition of at least 80% (in particular regarding phospholipid and surfactant proteins SP-B and SP-C) and it has a viscosity equal to or less than 15 mPas (cP) at room temperature when it is suspended in an aqueous solution at a concentration of 80/mg/ml. The viscosity can be determined according to known methods.

With the term “blender” it is meant an apparatus, typically in stainless steel, used to mix, crush, mince organic substances. It typically consists of container with a rotating metal blade at the bottom, powered by an electric motor.

By the term “sterile” it is meant a product which meets the criteria of sterility according to the European Pharmacopoeia (Ph. Eur. 1998, Chapters 2.6.1 and 5.1.1). Further regulations for sterility of the final product include the US Pharmacopoeia 23/NF18, 1995, pp. 1686-1690 and 1963-1975.

Surfactant activity” for a surfactant preparation is defined as the ability to lower the surface tension.

The in vitro efficacy of exogenous surfactant preparations is commonly tested by measuring its capability of lowering the surface tension using suitable apparatus such as Wilhelmy Balance and Captive Bubble Surfactometer.

The in vivo efficacy of exogenous surfactant preparations is commonly tested by measuring two parameters:
i) the tidal volume which is an index of the lung compliance and
ii) the lung gas volume which is an index of the alveolar air expansion or patency at the end of expiration, and hence of the capability of forming a stable phospholipid film in the alveoli at the end of expiration.

### DETAILED DISCLOSURE OF THE INVENTION

The characteristics of the process of the invention, the product obtained, and the corresponding pharmaceutical compositions will be described in the following detailed description.

In a first embodiment, the invention provides a process for the preparation of an exogenous pulmonary surfactant selected from modified natural surfactants or reconstituted synthetic surfactants, comprising the steps of:
i) extracting said modified natural surfactant from mammalian lung in form of dry paste or mixing the dry components of said reconstituted synthetic surfactant;
ii) purifying the obtained paste or mixture;
iii) wherein the step ii) of purification is performed by:
iv) dissolving the paste of the modified natural surfactant or the components of the reconstituted surfactant in an organic solvent comprising 2-methyl-2-propanol;
v) subjecting the obtained solution to sterilization by filtration; and
vi) drying.

Advantageously the paste of the modified natural surfactant could be prepared according to methods reported in the art, for example by methods based on elution on a chromatographic column or bronchoalveolar lavage.

Preferably the organic solvent comprises 2-methyl-2-propanol, where a solvent consisting of 2-methyl-2-propanol only is even more preferred More in a further preferred embodiment, the solvent is anhydrous 2-methyl-2-propanol, with a purity higher than 99.5%.

The dissolution of step iii) of the process of the present invention is usually conducted by applying a gentle warming, i.e. a temperature comprised between 30° and 40° C., preferably at 35° C.±1° C. under stirring.

The skilled person in the art shall determine the rate and time of stirring, which is typically comprised between 300-400 rpm for a time of 15-30 minutes.

The skilled person in the art shall determine the ratio between the surfactant and the organic solvent. In a preferred embodiment of the invention said ratio is higher than 5% w/w. It has indeed been found that lower percentages could not provide a complete dissolution.

Preferably, it is comprised between 6 and 20% w/v, more preferably 8-12% w/v.

The sterilization by filtration of step iv) of the process of the present invention could be carried out according to the knowledge of the skilled person in the art.

Optionally, the solution is subjected to a clarification step through a 0.45 micron pore size membrane filter; and afterwards it is sterilized by filtration, for example with filters of 0.22, and 0.1 micron pore size.

In step v) of the process of the present invention, the solvent could be removed according to methods known to the skilled person, for example by dialysis or evaporation under nitrogen and/or exposure to vacuum or by other appropriate techniques such as lyophilisation.

Since the chemical and physical properties of the 2-methyl-2-propanol as the solvent of the present invention, faster techniques of drying such as tangential flow filtration could advantageously be applied.

Of note, according to the present process, the amount of residual 2-methyl-2-propanol at the end of the drying step is equal to or even lesser than 3000 ppm, preferably equal to or lesser than 1500 rpm.

Any exogenous modified natural pulmonary surfactant or reconstituted pulmonary surfactant could be used. Advantageously, the reconstituted surfactant is selected from lucinactant Surfaxin™, Windtree Therapeutics, Inc., Warrington, Pa) and the surfactant quoted in the art as CHF 5633 (Chiesi Farmaceutici SpA, Italy), while the modified natural surfactant is selected from poractant alfa (Curosurf®, Chiesi Farmaceutici SpA, Italy) and biosimilars thereof, beractant (Survanta®, Abb Vie Inc, USA) and bovactant (Alveofact®, Lyomark Pharma GmbH, Germany), calfactant (Infasurf®, Ony Biotech, USA), Bles® (Bles Biochemicals Inc, Canada), KeLiSu (Double Crane, China), Surfactant TA (Surfaten®, Mitsubishi Tanabe Pharma Corporation, Tokyo, and other such as Surfacen® Beraksurf®, Newfactan®, Surfactant Gray®, and Surfactant BL®. A preferred modified natural surfactant is poractant alfa. Therefore, in a preferred embodiment, the invention is directed to a process for preparing poractant alfa, said process comprising the steps of:
i) mincing the pig lungs in a suitable blender;
ii) extracting the minced lungs with a physiological solution, then filtering and subjecting the mixture to centrifugation;
iii) extracting the supernatant with an organic solvent or mixtures thereof then evaporating the organic phase to dryness;
iv) separating the fractions containing the polar lipids and the hydrophobic proteins SP-C and SP-B;
v) collecting and pooling said fractions then evaporating the organic solution to dryness to yield the surfactant in the form of dry paste;
vi) dissolving the paste of the modified natural surfactant in an organic solvent comprising 2-methyl-2-propanol;
vii) subjecting the obtained solution to sterilization by filtration; and
viii) drying.

Preferably the organic solvent only consists of 2-methyl-2-propanol.

As further described in detail in the below experimental part with reference to Examples 3 and 4, the use of 2-methyl-2-propanol allows obtaining a product exhibiting the same quali-quantitative composition and exhibiting tidal volumes similar to those of poractant alfa control batch.

The step i) could be carried according to the knowledge of the skilled person in the art.

In the above step ii) the minced lungs are extracted with a physiological solution. The mixture is filtered through a strainer and subjected to centrifugation at advantageously 1,000×g at 20° C. for less than 30 min, preferably for 15 min, to eliminate cell debris.

After cooling to 4° C. for 2 hours the supernatant is further re-centrifuged at 3,000×g, advantageously for not more than 2 hours, preferably for not more than one hour, more preferably for 15 min.

In the above step iii) the raw solid surfactant in the form of pellets is removed and extracted with a mixture of a halogen hydrocarbon and a short chain aliphatic alcohol as disclosed in EP 286,011.

The obtained solution is filtered and washed with water, then evaporated to dryness under vacuum using equipments well-known to the skilled person.

According to step iv), the separation of the fractions containing the components responsible of the biological activity (polar lipids, mainly phospholipids, and hydrophobic proteins SP-C and SP-B) from the other fractions by extraction could be carried out according to methods known in the art. (Saini R K et al Int J Molecular Sci 2021, 22, 1-19).

In case of poractant alfa, advantageously, the separation of the fractions containing the components responsible of the biological activity (polar lipids, mainly phospholipids, and hydrophobic proteins SP-C and SP-B) from the other fractions (step iv) is carried out by size-exclusion gel-chromatography using a lipophilic Sephadex derivative as stationary phase, and a mixture of a halogen hydrocarbon and a short chain aliphatic alcohol as eluant as reported in EP 286,011.

More advantageously the stationary phase used for filling the chromatographic column is constituted of Sephadex®, sold under the trademark of Lipidex®-5000 by different suppliers such as Sigma Co and Packard Ins.

Alternatively, step iv) could be performed using methods that do not employ chlorinated solvents such as chloroform.

For example, step iv) could be carried out using supercritical fluids as described in EP 670846. Further tuning of the inert support, pressure and temperature of the supercritical fluids, and type and amount of the cosolvent could be performed by the skilled person according to its knowledge.

Otherwise, said step could be performed by first removing components such as cholesterol and mono-, di- and triglycerides by nanofiltration and ultrafiltration according to methods known in the art (C. Allegre et al./Journal of Membrane Science 269 (2006) 109-117) then obtaining the components responsible of the biological activity by ion-exchange-chromatography and a mixture of t-butanol and water as eluant Separation of lipid mixtures, 1972. Lab. Tech. Biochem. Mol. Biol. 3, 393-469.

In the above step v) the fractions containing the polar lipids and the hydrophobic proteins SP-B and SP-C are collected and pooled, then the organic solution is evaporated to dryness at a temperature of less than 50° C., preferably less than 40° C.

The above steps vi)-viii), which are carried out as disclosed above are particularly critical for poractant alfa.

Said modified natural surfactant is indeed endowed with peculiar properties in terms of viscosity in comparison to the other ones currently available.

According to the prior art, this is due to the fact that poractant alfa is essentially constituted of the components deemed responsible of the biological activity, i.e. polar lipids and the hydrophobic proteins SP-C and SP-B as well as that the phospholipids fraction is enriched of two components, i.e. plasmalogens and polyunsaturated fatty acids containing PLs (see Rüdiger M et al. Am J Physiol Lung Cell Mol Physiol 2005, 288, 379-383).

Therefore, the chosen solvent and conditions shall guarantee that following steps vi) and vii) the composition of the surfactant is not altered.

The paste of poractant alfa at the end of step viii) has the following composition (all the values are expressed as percentages on the total weight of the dry mass of the surfactant):
total phosphorus content (P): 3.6-4.2%;
hydrophobic proteins SP-C and SP-B: 0.5-2.2%;
neutral lipids: equal to or less than 0.5%;
cholesterol: equal to or less than 0.5%;
free fatty acids (FFAs): equal to or less than 1.5%, preferably less than 1.0%
residual solvents: equal to or less than 6.0%;
As far as phosphatidylcholine species are concerned, surfactant has also the following percentage composition based on the total phosphorous content:
phosphatidylcholine (PC) content: 66-77%;
dipalmitoyl phosphatidylcholine (DPPC) content: higher than 28%, preferably 33-45%;
lysophspatidylcholine (LPC) content: equal to or less than 2.0%

Optionally, the dry paste may contain small amounts of components which are unessential for the biological activities such neutral lipids, cholesterol, free fatty acids, carbohydrates and residual solvents.

Advantageously, the total amount of said unessential components is less than 5% calculated on the total weight of the dry mass of the surfactant, preferably less than 3.5%, more preferably less than 2.5%, even more preferably less than 1%.

The various components of the pulmonary surfactants could be determined according to methods reported in the art.

For example, the total phosphorus content could be estimated according to a colorimetric procedure based on the method of Barlett G R J Biol Chem, 1959, 234, 406-408.

The content of PC and LPC could be estimated according to the following procedure.

First, the phospholipidic classes of the surfactant are first separated by two dimensional thin-layer chromatography (TLC) according to Poorthius B J H et al J Lipid Res 1976, 17, 433-436.

The silica corresponding to PC and LPC spots (identified by a comparison with reference standards), are recovered by the TLC plate with an opportune blade and transferred to a test tube.

The samples mineralization is carried out in each test tube, according to the procedure reported for the determination of the total phosphorus content (vide supra).

The content of DPPC could be estimated with OsO₄ according to the method reported in Mason R J J Lipid Res 1976, 17, 281-284.

The phosphorus content related to the DPPC is determined with respect to the total phosphorus content.

The content of free fatty acids, neutral lipids (triglycerides, diglycerides and monoglycerides) and cholesterol (fractionated free cholesterol and cholesterol esters) could be estimated by HPLC analysis according to methods known in the art.

The content of the hydrophobic proteins SP-C and SP-B could be determined using a commercially available kit “BCA Protein Assay Reagent” according to the following procedure. Otherwise, it may be determined according to HPLC or capillary electrophoresis methods reported in the art.

Carbohydrates could be determined in accordance with the procedure described by Dubois et al Anal Chem 1956, 28, 350-356 and expressed in terms of glucose.

The residual solvents and the residual water content are estimated by gas-chromatography and Karl-Fisher method, respectively according to procedures well-known to the person skilled in the art.

The present invention is also directed to a modified natural surfactant extracted from mammalian lung in the form of paste or to a reconstituted surfactant as obtained, or obtainable, by the process of the invention.

The present invention also concerns pharmaceutical compositions comprising a modified natural surfactant extracted from mammalian lung in the form of paste or to a reconstituted surfactant as obtained or obtainable by the process of the invention.

Said compositions are advantageously administered in the form of a solution, dispersion, suspension or as dry powder. Preferably said compositions are in form of sterile formulations wherein the pulmonary surfactant is suspended in a suitable solvent or resuspension medium, typically physiological sodium chloride aqueous solution.

Therefore, in order to prepare a sterile formulation in form of suspension, the process of the invention optionally comprises the following steps:
ix) re-suspending the dried product in a sterile physiological sodium chloride aqueous solution at room temperature under agitation;
x) optionally, adjusting the pH of the suspension to the desired value; and
xi) filling the suspension into suitable single-use vials under aseptic conditions.

The step ix) is known in the art as thin-film hydration and it could be accomplished by mechanical stirring or by sonication, and optionally followed by extrusion.

Advantageously the concentration of the pulmonary surfactant is in the range of from about 2 to about 160 mg of surfactant per ml, preferably between 10 and 100 mg/ml, more preferably between 20 and 80 mg/ml. When poractant alfa is used, the preferred concentration is 80 mg/ml.

The sterile formulations comprising the pulmonary surfactant as obtained by the process of the invention are administered in manners known to the person skilled in the art, preferably by intratracheal installation (infusion or bolus) or by nebulisation, and they are useful for the treatment or prophylaxis of a variety of pulmonary disorders, abnormal conditions and diseases caused or related to a deficiency or dysfunction of the pulmonary surfactant. Examples are hyaline membrane disease, infant and adult respiratory distress syndromes (IRDS and ARDS), acute lung injury (such as that resulting from ozone inhalation, smoke inhalation or near drawing), conditions of surfactant inactivation triggered by volutrauma and barotrauma, meconium aspiration syndrome, capillary leak syndrome, bacterial and viral pneumonia, and bronchopulmonary dysplasia.

They could also be useful for the treatment or prophylaxis of other respiratory disorders such as pneumonia, bronchitis, COPD (chronic obstructive pulmonary disease), asthma, and cystic fibrosis as well as for the treatment of serous otitis media (glue ear).

The following examples illustrate in detail the invention.

### Examples

### Example 1—Preparation of Poractant Alfa Dry Paste

About 180 kg of pig lungs are triturated in a blender, and the tissue fragments are washed in a physiological solution. The mixture is filtered through a strainer and subjected to preliminary centrifugation at 1000×g at 20° C. for 15 min, to eliminate cell debris. The supernatant liquor is then re-centrifuged at 3000×g at 4° C. for 2 hours. The raw surfactant is removed and extracted with chloroform:methanol 2:1 (v/v), filtered, washed with water and the organic phase is evaporated thus obtaining a raw lipid extract. The lipid fraction extract (about 500 g) is recovered with about 4 litres of chloroform:methanol 1:4 (v/v), filtered through a 2.5 micron membrane filter insufflating nitrogen at 0.2-0.4 atmospheres, then separated by reversed-phase chromatography using a column filled with the LipidexR-5000 packing material 2 with chloroform:methanol 1:4 (v/v) as eluant.

About 200 g of purified poractant alfa as dry material was obtained and tested using the analytical methods reported above.

The purified poractant alfa dry material has the following composition (all the values are expressed as percentages on the total weight of the dry mass):
total phosphorous content: 3.8%;
phosphatidylcholine (PC) content: 68%;
dipalmitoyl phosphatidylcholine (DPPC) content: 40%;
lysophspatidylcholine (LPC) content: 1.0%
hydrophobic proteins SP-C and SP-B: 1.6%;
free fatty acids: 0.9%.
neutral lipids: none detectable;
cholesterol: none detectable;
carbohydrates: none detectable.

### Example 2—Determination of the Suitable Solvent for Sterilization by Filtration

The evaluation included the alcohols chemical group, namely ethanol, 1-propanol, 2-propanol and 2-methyl-2-propanol (tert-butanol).

As a first screening test, equal quantities of poractant alfa paste (about 10 grams) was diluted and mixed up with 100 mL of each alcohol. In any case, a slight heating (35° C.) was applied to promote the dissolution and to avoid precipitation phenomena.

The relevant appearance was then visually assessed and compared with a reference solution of poractant alfa in chloroform. Only tert-butanol provided a limpid solution among the alcohols tested, like the chloroform reference. The other alcohols gave rise to opalescent or very turbid and non-homogeneous solution.

Once defined the tert-butanol as alternative solvent, it was decided to move to the filtration step, in order to understand the product behavior during that unit operation.

The following filtering system was used:
A glass fiber prefilter, having 0.45 μm pore size and approximately 17 cm² of filtration surface.
A PVDF bioburden reduction filter, 0.22 μm pore size, having filtration surface of approximately 17 cm².
A PVDF sterilizing filter, 0.1 μm pore size and filtration surface of approximately 17 cm².

The filtration was carried out through a peristaltic motion and with a constant pressure monitoring to calculate the ΔP between upstream and downstream sides of the membranes.

For sake of comparison, the same trial was undergone on both a t-butanol and a chloroform solution.

No criticalities were observed during the process.

The obtained solution was dried using a rotating evaporator (Rotavapor®) according to the following parameters:
Temperature of heating bath: 35° C.
Temperature of the chiller: 15-17° C.
Vacuum: 45-50 mbar
Rotation: 100 rpm

A solid paste was collected and resuspended with a sterile solution of Sodium Chloride 0.9% w/v in water. The final suspension was filled into vials and stored at 2-8° C.

### Example 3—Product Characterization

Based on the process described above, a pilot batch was prepared to evaluate the general product feasibility as well as the behavior of the main Critical Quality Attributes of the Curosurf®.

The dried paste was re-suspended in a sterile physiological sodium chloride aqueous solution and mixed inv the Rotavapor®.

The suspension thus obtained was distributed in vials use in a concentration of 80 mg of surfactant per ml of physiological sodium chloride aqueous solution.

The vials were undergone to analytical testing to verify whether the current specifications of the product were still met despite the amendments applied to its manufacturing process.

The results met all the specification criteria.

The batch was also tested for morphological imaging for the particle characterization and compared with a reference batch produced in standard condition, i.e. through the current process involving chloroform as process solvent.

Curosurf® as obtained by the process of the invention turned out to be very similar in terms of micellar dimension, and micellar shape to the Curosurf® control batch.

### Example 4—In Vivo Characterization

To determine the surfactant activity, the effects of intratracheal administration of the batch obtained by the process of the invention on tidal volume, was evaluated by in-vivo testing on premature newborn rabbits.

A Curosurf® control batch was used.

The surfactant preparations were administered at a standard dose of 2.5 ml/kg.

The immature newborn rabbits were ventilated in parallel with a standardized sequence of peak insufflation pressures. To open up the lungs, pressure is first set at 35 cmH₂O for 1 min. After this recruitment manoeuvre, pressure is lowered to 25 cmH₂O for 15 min and further on to 20 and 15 cm H₂O.

Finally, pressure is raised again to 25 cmH₂O for 5 min, after which the lungs are ventilated for additional 5 min with nitrogen.

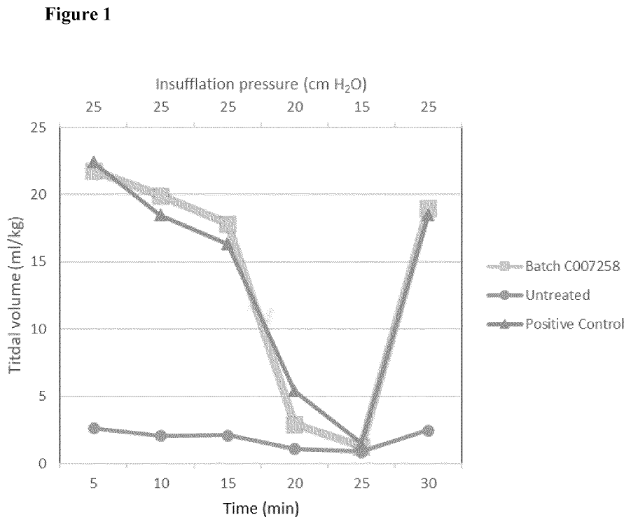

The tidal volumes, expressed as ml/kg, are measured and the results, given as median values, are reported in FIG. 1.

It can be appreciated that animals treated with Curosurf® as obtained by the process of the invention show tidal volumes similar to those obtained with Curosurf® control batch.

1. A process for the preparation of an exogenous pulmonary surfactant selected from the group consisting of a modified natural surfactant or a reconstituted synthetic surfactant, comprising:
i) extracting said modified natural surfactant from mammalian lung in form of dry paste or mixing the dry components of said reconstituted synthetic surfactant;
ii) purifying the obtained paste or mixture;
iii) wherein the step ii) of purification is performed by:
iv) dissolving the paste of the modified natural surfactant or the components of the reconstituted surfactant in an organic solvent comprising 2-methyl-2-propanol,
v) subjecting the obtained solution to sterilization by filtration; and
vi) drying.

2. The process according to claim 1, wherein the organic solvent only consists of 2-methy-2-propanol.

3. The process according to claim 1, wherein said step i) is performed at a temperature comprised between 30 and 40° C. under stirring.

4. The process according to claim 3, wherein the stirring is performed at a rate of 300-400 rpm for a time of 15-30 minutes.

5. The process according to claim 1, wherein the ratio between the surfactant and the organic solvent in step 1 is from 6 to 20% w/v.

6. The process according to claim 1, wherein the reconstituted surfactant is lucinactant or elifactant.

7. The process according to claim 1, wherein the modified natural surfactant is selected from the group consisting of poractant alfa and biosimilar thereof, beractant, calfactant and bovactant.

8. The process according to claim 7, wherein the modified natural surfactant is poractant alfa.

9. The process according to claim 6, which comprises:
i) mincing pig lungs in a suitable blender;
ii) extracting the minced lungs with a physiological solution, then filtering and subjecting the mixture to centrifugation;
iii) extracting the supernatant with an organic solvent or mixtures thereof then evaporating the organic phase to dryness;
iv) separating the fractions containing the polar lipids and the hydrophobic proteins SP-C and SP-B;
v) collecting and pooling said fractions then evaporating the organic solution to dryness to yield the surfactant in the form of dry paste;
vi) dissolving the paste of the modified natural surfactant in an organic solvent comprising 2-methyl-2-propanol;
vii) subjecting the obtained solution to sterilization by filtration; and
viii) drying.

10. The process according to claim 9, wherein said separating iv) is performed by size-exclusion liquid-gel chromatography.

11. The process according to claim 9, wherein said separating iv) is performed without the use of chlorinated solvents.

12. The process according to claim 11, wherein said separating iv) is performed by using a supercritical fluid.

13. The process according to claim 1, further comprising:
ix) re-suspending the dried product in a sterile physiological sodium chloride aqueous solution at room temperature under agitation;
x) optionally, adjusting the pH of the suspension to the desired value; and
xi) filling the suspension into suitable single-use vials under aseptic conditions.

14. An exogenous pulmonary surfactant as obtained by the process of claim 1.

15. A pharmaceutical formulation comprising the exogenous pulmonary surfactant obtained by the process of claim 1.

16. A method for the prevention or treatment of a pulmonary disorder, or an abnormal condition or disease caused by or related to a deficiency or dysfunction of a pulmonary surfactant, comprising administering to a subject in need thereof and effective amount of an exogenous pulmonary surfactant as obtained by the process of claim 1.

17. The method according to claim 16, wherein said pulmonary disorder, or an abnormal condition or disease caused by or related to a deficiency or dysfunction of a pulmonary surfactant is neonatal respiratory distress syndrome (RDS).
